(19) United States
(12) Patent Application Publication
(10) Pub. No.: US 2026/0207941 A1
(43) Pub. Date: Jul. 23, 2026

(54) HYBRID RETINAL STIMULATION DEVICE, OPERATING METHOD THEREOF, AND RECORDING MEDIUM

(71) Applicants: Industry-Academic Cooperation Foundation, Dankook University, Gyeonggi-do (KR); KOREA INSTITUTE OF SCIENCE AND TECHNOLOGY, Gyeonggi-do (KR)

(72) Inventors: Jae-Hyoung Park, Gyeonggi-do (KR); Seung-Ki Lee, Seoul (KR); Yong-Kweon Kim, Seoul (KR); Seung-Han Chung, Seoul (KR); Maesoon Im, Seoul (KR); Chaesung Kim, Seoul (KR)

(73) Assignees: Industry-Academic Cooperation Foundation, Dankook University, Gyeonggi-do (KR); KOREA INSTITUTE OF SCIENCE AND TECHNOLOGY, Gyeonggi-do (KR)

(21) Appl. No.: 19/452,375

(22) Filed: Jan. 18, 2026

(30) Foreign Application Priority Data
Jan. 20, 2025 (KR) 10-2025-0008091
Apr. 1, 2025 (KR) 10-2025-0042332

(51) Int. Cl. A61N 1/36; A61N 1/05; G16H 20/40; G16H 40/67

(52) U.S. Cl. CPC A61N 1/36046; A61N 1/0543; G16H 20/40; G16H 40/67

(57) ABSTRACT

A hybrid retinal stimulation device includes a glass substrate in which a through hole is formed, a multi-channel stimulation electrode arranged in the through hole of the glass substrate and configured to provide electrical stimulation to the retinal cells, a multi-channel recording electrode arranged in a through hole of the glass substrate to measure a neural response signal generated from the retinal cells while arranged at a position spaced apart by a determined distance from the multi-channel stimulation electrode, a micro light-emitting diode (LED) array arranged in a lower portion of the glass substrate and configured to provide optical stimulation to the retinal cells from between the multi-channel stimulation electrode and the multi-channel recording electrode, and a controller configured to control synchronization between the electrical stimulation and the optical stimulation by independently controlling an operation of the multi-channel stimulation electrode and the micro LED array.

### CROSS-REFERENCE TO RELATED APPLICATION

This application claims the benefit of Korean Patent Application No. 10-2025-0008091, filed on January 20, 2025, and 10-2025-0042332, filed on April 1, 2025, in the Korean Intellectual Property Office, the entire disclosures of which are incorporated herein by reference for all purposes.

### BACKGROUND

### 1. Field of the Invention

One or more embodiments relate to a hybrid retinal stimulation device that stimulates retinal cells by combining electrical stimulation with optical stimulation, and an operating method thereof.

### 2. Description of the Related Art

Retinal degenerative diseases such as age-related macular degeneration (AMD) or retinitis pigmentosa (RP) may result in gradual loss of photoreceptor cells within the retina, leading to progressive vision loss and eventually to blindness.

To treat these retinal degenerative diseases, electrical stimulation-based retinal prosthetics and optogenetics technologies have been studied. Electrical stimulation-based retinal prosthetics is technology that induces artificial vision by attaching electrodes to the retina and applying electrical stimulation to the retina. The technology has limitations with regard to spatial resolution due to interference between stimulating electrodes, which may cause difficulty in restoring high-resolution vision.

In addition, optogenetics is technology that involves expressing photoreceptor proteins in retinal cells and irradiating the retinal cells with light of a determined wavelength to induce neural activity. This requires high-intensity light for effective stimulation, which carries the risk of retinal tissue damage.

The foregoing information may be provided as related art for the purpose of facilitating an understanding of the present disclosure. No claim or determination is made as to whether any of the above description is applicable as prior art related to the present disclosure.

### SUMMARY

Embodiments provide a hybrid retinal stimulation device that precisely synchronizes electrical stimulation and optical stimulation to retinal cells, enabling more efficient and safe retinal stimulation, and an operating method thereof.

However, technical aspects are not limited to the foregoing aspect, and there may be other technical aspects.

According to an aspect, there is provided a hybrid retinal stimulation device including a glass substrate in which a through hole for providing electrical stimulation and optical stimulation to retinal cells is formed, a multi-channel stimulation electrode arranged in the through hole of the glass substrate and configured to provide the electrical stimulation to the retinal cells, a multi-channel recording electrode arranged in a through hole of the glass substrate to measure a neural response signal generated from the retinal cells while arranged at a position spaced apart by a determined distance from the multi-channel stimulation electrode, a micro light-emitting diode (LED) array arranged in a lower portion of the glass substrate and configured to provide the optical stimulation to the retinal cells from between the multi-channel stimulation electrode and the multi-channel recording electrode, and a controller configured to control synchronization between the electrical stimulation and the optical stimulation by independently controlling an operation of the multi-channel stimulation electrode and the micro LED array.

The controller may be implemented based on a field-programmable gate array (FPGA) and configured to operate as a master device.

The controller may be configured to communicate with one or more slave devices, using serial peripheral interface (SPI) communication, and independently control the stimulation electrode providing the electrical stimulation to the retinal cells or the micro LED array providing the optical stimulation to the retinal cells.

The controller may be configured to control at least one of an intensity, a frequency, a pulse width, or a duration time of the electrical stimulation by analyzing a neural response signal measured through the recording electrode.

The controller may be configured to control at least one of an intensity, a blinking cycle, or a duration time of the optical stimulation by analyzing a neural response signal measured through the recording electrode.

The multi-channel stimulation electrode and the multi-channel recording electrode may be formed by forming a pillar-shaped vertical through structure on a silicon substrate and bonding a glass-based dielectric on the silicon substrate on which the vertical through structure is formed, through a deep silicon (Si) etching process and an anodic bonding process, respectively, generating a glass substrate by filling an empty area of the silicon substrate with the dielectric after lowering viscosity of the dielectric by heating the dielectric, and removing the dielectric remaining after filling the empty area based on an upper surface of the silicon substrate, through a glass reflow process and a chemical mechanical polishing (CMP) process, respectively, forming a micro-pillar structure by selectively etching a partial area of the silicon substrate through the deep silicon etching process while the silicon substrate is arranged in an inverted orientation, forming a pointed electrode structure by gradually etching exposed silicon regions of the micro-pillar structure through a wet etching process, and forming a metal pattern on the pointed electrode structure through a metal patterning process, and arranging electrodes on a lower surface of the glass substrate and performing dicing through an electrode arrangement process and a dicing process, respectively.

The micro LED array may be formed by patterning a metal layer on a substrate according to a predetermined electrode structure and subsequently adding an insulating layer between the patterned metal layer through a metal patterning process and an insulation process, respectively, depositing a chromium (Cr) layer and a gold (Au) layer on top of the metal layer and the insulating layer through a sputtering process, coating a photosensitive agent on top of the chromium layer and the gold layer and subsequently plating a copper (Cu) layer thereon through a copper electroplating process, forming a solder bump on top of the copper layer through a solder bump electroplating process, and placing an individual micro LED onto the solder bump through a pick & place process.

According to an aspect, there is provided an operating method of a hybrid retinal stimulation device including transmitting a control command according to a predetermined stimulation protocol to one or more slave devices via a controller implemented based on a field-programmable gate array (FPGA) and configured to operate as a master device, providing electrical stimulation or optical stimulation to retinal cells through a multi-channel stimulation electrode or a micro light-emitting diode (LED) activated by the control command transmitted to the one or more slave devices, collecting, through a recording electrode, a neural response signal generated by the provided electrical stimulation or the provided optical stimulation, and retransmitting to the one or more slave devices, via the controller, a control command for controlling at least one of the electrical stimulation or the optical stimulation by analyzing the collected neural response signal.

The retransmitting of the control command may include controlling at least one of an intensity, a frequency, a pulse width, or a duration time of the electrical stimulation by analyzing a neural response signal measured through the recording electrode.

The retransmitting of the control command may include controlling at least one of an intensity, a blinking cycle, or a duration time of the optical stimulation by analyzing a neural response signal measured through the recording electrode.

A non-transitory computer-readable storage medium may store instructions that, when executed by one or more processors, cause the one or more processors to perform the operating method.

Additional aspects of embodiments will be set forth in part in the description which follows and, in part, will be apparent from the description, or may be learned by practice of the disclosure.

According to embodiments, a high level of neural activation may be induced even at a lower stimulation intensity by providing synchronized electrical stimulation and optical stimulation to retinal cells.

### BRIEF DESCRIPTION OF THE DRAWINGS

With regard to the description of the drawings, the same or similar reference numerals may be used to refer to the same or similar components.

These and/or other aspects, features, and advantages of the invention will become apparent and more readily appreciated from the following description of embodiments, taken in conjunction with the accompanying drawings of which:

FIG. 1 is a diagram schematically illustrating an overall configuration of a hybrid retinal stimulation device according to an embodiment;

FIG. 2 is a diagram illustrating an actual implementation form of a hybrid retinal stimulation device, according to an embodiment;

FIG. 3 is a diagram illustrating an electrode formation process of a hybrid retinal stimulation device, according to an embodiment;

FIG. 4 is a diagram illustrating a micro light-emitting diode (LED) array formation process of a hybrid retinal stimulation device, according to an embodiment;

FIG. 5 is a block diagram illustrating a control system of a hybrid retinal stimulation

device, according to an embodiment;

FIG. 6 is a flowchart illustrating a method of providing hybrid stimulation of a hybrid retinal stimulation device, according to an embodiment; and

FIG. 7 is a diagram illustrating a controller included in a hybrid retinal stimulation device, according to an embodiment.

### DETAILED DESCRIPTION

The following detailed structural or functional description is provided as an example only and various alterations and modifications may be made to the embodiments. Thus, an actual form of implementation is not construed as limited to the embodiments described herein and should be understood to include all changes, equivalents, and replacements within the idea and the technical scope of the disclosure.

As used herein, each of phrases such as "A or B," "at least one of A and B," "at least one of A or B," "A, B, or C," "at least one of A, B, and C," "at least one of A, B, or C," and "one or a combination of at least two of A, B, and C" may include any one of the items listed together in the corresponding one of the phrases or all possible combinations thereof. Although terms, such as first, second, and the like are used to describe various components, the components are not limited to the terms. These terms should be used only to distinguish one component from another component. For example, a first component may be referred to as a second component, and similarly, the second component may also be referred to as the first component.

It should be noted that when one component is described as being "connected," "coupled," or "joined" to another component, the first component may be directly connected, coupled, or joined to the second component, or a third component may be between the first and second components.

The singular forms "a," "an," and "the" used herein are intended to include the plural forms as well, unless the context clearly indicates otherwise. It will be further understood that the terms "comprises/comprising" and/or "includes/including," when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical and scientific terms have the same meanings as those commonly understood by one of ordinary skill in the art to which this disclosure pertains. Terms such as those defined in commonly used dictionaries are to be interpreted as having a meaning that is consistent with their meaning in the context of the relevant art and are not to be interpreted in an idealized or overly formal sense unless expressly so defined herein.

Hereinafter, the embodiments are described in detail with reference to the accompanying drawings. When describing the embodiments with reference to the accompanying drawings, like reference numerals refer to like components and a repeated description related thereto is omitted.

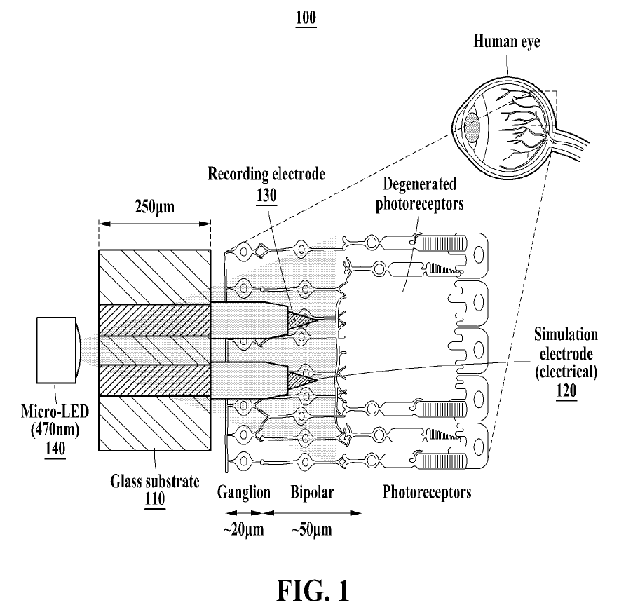

FIG. 1 is a diagram schematically illustrating an overall configuration of a hybrid retinal stimulation device according to an embodiment.

A hybrid retinal stimulation device 100 may provide a method of reactivating a visual signal by inducing a neural response in remaining retinal cells by providing a combined electrical stimulation and optical stimulation, even when the function of some photoreceptors is lost due to retinal diseases.

Referring to FIG. 1, the hybrid retinal stimulation device 100 may include at least one of a glass substrate 110, a stimulation electrode 120, a recording electrode 130, a micro light-emitting diode (LED) 140, or a controller (not shown).

First, the glass substrate 110 may provide a physical basis for transmitting electrical stimulation and optical stimulation to the retinal cells (e.g., a ganglion cell and a bipolar cell). The glass substrate 110 may be formed of a material with excellent optical transmittance, and the stimulation electrode 120 and the recording electrode 130 may be arranged therein through a through hole.

The stimulation electrode 120 may be arranged in a through hole of the glass substrate 110 to directly provide electrical stimulation to retinal cells. The stimulation electrode 120 may be configured as multi channels, and operations thereof may be individually controlled through a multi-channel stimulation generator that operates as a slave device.

The recording electrode 130 may be arranged in the through hole while spaced apart by a determined distance from the stimulation electrode 120 to measure a neural response signal generated from the retinal cells. The recording electrode 130 may also be configured as multi channels and may quantitatively analyze a neural response to electrical or optical stimulation.

The micro LED 140 may be arranged on a lower portion of the glass substrate 110 and may provide optical stimulation to the retinal cells from between the multi-channel stimulation electrode 120 and the multi-channel recording electrode 130. The micro LED 140 may be implemented in the form of a micro LED array corresponding to the number of the stimulation electrode 120 or the recording electrode 130, and operations thereof may be individually controlled through an LED matrix controller that operates as a slave device.

The controller may control synchronization between the electrical stimulation and the optical stimulation by independently controlling an operation of the multi-channel stimulation electrode 120 and the micro LED 140. The controller may be implemented based on a field-programmable gate array (FPGA) to operate as a master device and may use a serial peripheral interface (SPI) protocol to communicate with one or more slave devices.

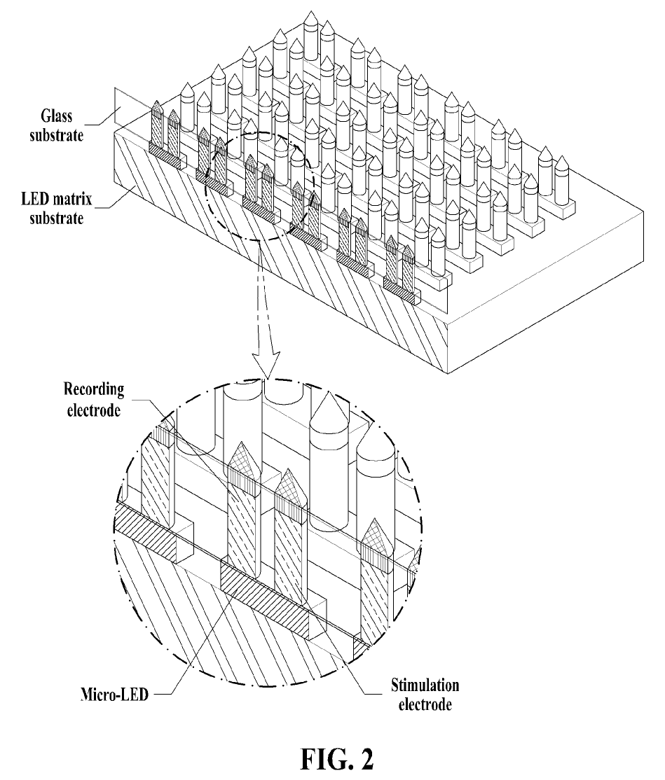

FIG. 2 is a diagram illustrating an actual implementation form of a hybrid retinal stimulation device, according to an embodiment.

Referring to FIG. 2, the hybrid retinal stimulation device (e.g., the hybrid retinal stimulation device 100 of FIG. 1) may provide electrical stimulation to retinal cells by a stimulation electrode (e.g., the stimulation electrode 120 of FIG. 1) arranged in a through hole formed in a glass substrate (e.g., the glass substrate 110 of FIG. 1). In addition, the hybrid retinal stimulation device may measure neural response information generated from the retinal cells by a recording electrode (e.g., the recording electrode 130 of FIG. 1) arranged in a through hole formed in the glass substrate.

The hybrid retinal stimulation device may have a micro LED arranged on a lower portion of the glass substrate. The micro LED may be arranged on an LED matrix substrate arranged under the glass substrate and may be arranged one by one between the stimulation electrode and the recording electrode.

By this alignment structure, the hybrid retinal stimulation device may provide hybrid stimulation, which provides electrical stimulation and optical stimulation to a same area simultaneously.

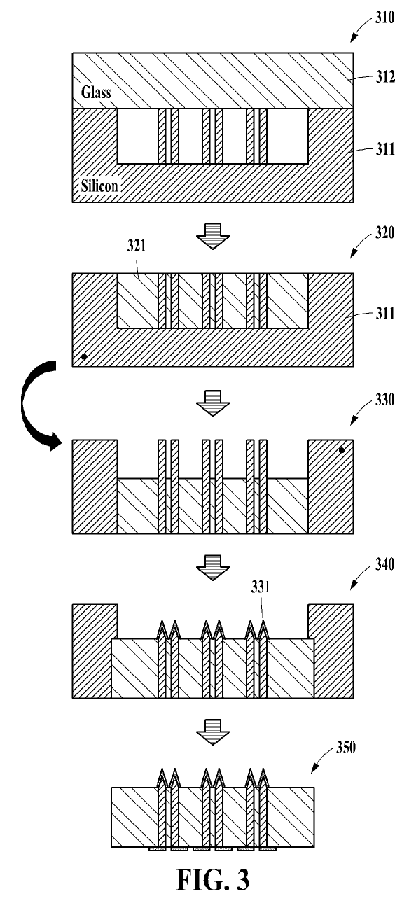

FIG. 3 is a diagram illustrating an electrode formation process of a hybrid retinal stimulation device, according to an embodiment. In an embodiment, at least one of the operations of FIG. 3 may be performed simultaneously or in parallel with another, and the order of the operations may be changed. In addition, at least one of the operations may be omitted, or another operation may be additionally performed. Each operation of the electrode formation process of the hybrid retinal simulation device illustrated in FIG. 3 may be performed by at least one processor included in a separate device for manufacturing the hybrid retinal stimulation device.

In operation 310, the processor may form a pillar-shaped vertical through structure in a silicon substrate 311 through a deep silicon (Si) etching process (e.g., deep reactive ion etching (DRIE)). The vertical through structure formed here may become an area, which a glass-based dielectric 312 may be aligned with and the electrode may be inserted into.

Thereafter, through an anodic bonding process, the processor may align and bond the glass-based dielectric 312 on the silicon substrate 311, on which the vertical through structure may be formed. Here, the processor may secure alignment accuracy between the etched vertical through structure and the glass-based dielectric 312 by utilizing an alignment mark during the anodic bonding process.

In operation 320, the processor may fill an empty area of the silicon substrate 311 with the dielectric 312 by heating the dielectric 312 to lower the viscosity thereof through a glass reflow process, thereby generating a single, integral glass substrate 321. For example, after the anodic bonding process is completed, the processor may perform the glass reflow process under the condition of a high temperature environment of about 850°C and high vacuum (e.g., 5×10⁻⁴ torr).

Thereafter, the processor may secure precision of subsequent processes by removing the dielectric 312 remaining on a surface of the silicon substrate 311, using an upper surface of the silicon substrate 311 as a reference surface, through a chemical mechanical polishing (CMP) process, to planarize the glass substrate 321 after the glass reflow process.

In operation 330, after completion of the CMP process, the processor may arrange the silicon substrate 311 in an inverted orientation such that a bottom surface of the silicon substrate 311 serves as a processing surface, and may selectively etch a partial area of the silicon substrate 311 by performing a deep silicon etching process on the bottom surface of the silicon substrate 311 to form a micro-pillar structure. Here, the glass substrate 321 may function as an etch stop layer while the deep silicon etching process on the bottom surface of the silicon substrate 311 is in progress.

In operation 340, the processor may form a pointed electrode structure by performing a wet etching process on exposed silicon regions of the micro-pillar structure formed through operation 330, thereby gradually etching the exposed silicon regions of the micro-pillar structure. Here, the processor may perform the wet etching process using an etchant, which may be mixture of nitric acid (HNO₃) and hydrofluoric acid (HF).

More specifically, during the wet etching process using the etchant, which may be mixture of nitric acid (HNO₃) and hydrofluoric acid (HF), the exposed silicon regions may be gradually etched as oxidation of the exposed silicon regions and removal of an oxide layer repeatedly occur.

Here, the exposed silicon regions may be formed into a pointed shape under specific conditions, as the etching speed may vary depending on the direction of a silicon crystal.

Thereafter, the processor may form a metal pattern 331 on a pointed electrode structure through a metal patterning process. For example, the processor may form a metal layer by applying a metal material such as titanium (Ti) or gold (Au) to the pointed electrode structure by a sputtering or electroplating method.

In operation 350, the processor may provide an interface for a connection with an external driving circuit by arranging electrodes on a lower surface of the glass substrate 321 through an electrode arrangement process. Here, the electrodes may be individually arranged on each of the stimulation electrode and the recording electrode. Thereafter, the processor may be cut into a form of an individual electrode array chip through a dicing process and used in the hybrid retinal stimulation device.

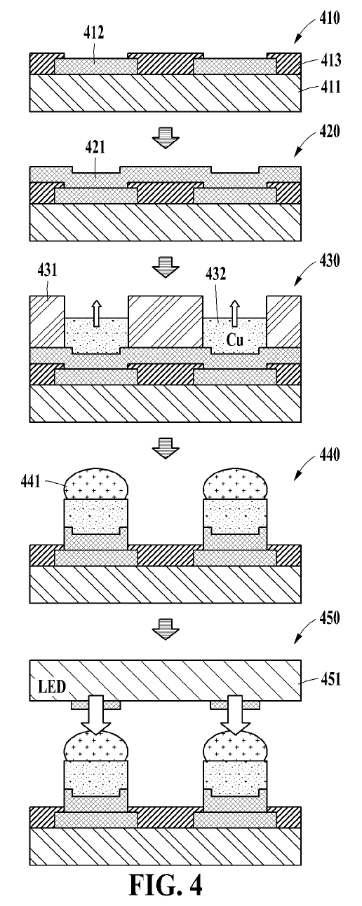

FIG. 4 is a diagram illustrating a micro LED array formation process of a hybrid retinal stimulation device, according to an embodiment. In an embodiment, at least one of the operations of FIG. 4 may be performed simultaneously or in parallel with another, and the order of the operations may be changed. In addition, at least one of the operations may be omitted, or another operation may be additionally performed. Each operation of the micro LED array formation process of the hybrid retinal stimulation device disclosed in FIG. 4 may be performed by at least one processor included in a separate device for manufacturing the hybrid retinal stimulation device.

In operation 410, the processor may pattern a metal layer 412 on a substrate 411 according to a predetermined electrode structure through a metal patterning process and an insulation process. Thereafter, the processor may form an insulating layer 413 between a patterned metal layers 412 to prevent electrical interference between electrodes.

In operation 420, the processor may deposit a chromium (Cr) thin film and a gold (Au) thin film 421 sequentially on top of the metal layer 412 and the insulating layer 413 through a sputtering process. Here, a chromium thin film may be used as an adhesive layer to strengthen adhesion, and a gold thin film 421 may be used to provide high conductivity and corrosion resistance properties.

In operation 430, the processor may enhance conductivity and durability of the electrode by coating a photosensitive agent 431 on top of the chromium thin film and the gold thin film 421 and subsequently plating a copper (Cu) layer 432 thereon through a copper electroplating process.

In operation 440, the processor may form a solder bump 441 on top of the copper layer 432 through a solder bump electroplating process. Subsequently, the solder bump 441 may function as a soldering interface for alignment and connection of a micro LED.

Finally, in operation 450, the processor may place an individual micro LED 451 onto the solder bump 441 through a pick & place process. This process may be performed by an automated robot, utilizing a high-resolution camera-based alignment system to position a micro LED in a precise position.

The micro LED array formed in this way may be arranged between a multi-channel stimulation electrode and a multi-channel recording electrode to provide optical stimulation to retinal cells in a same area as an area to which electrical stimulation may be provided by the stimulation electrode, thereby realizing a neural stimulation effect of a hybrid stimulation method.

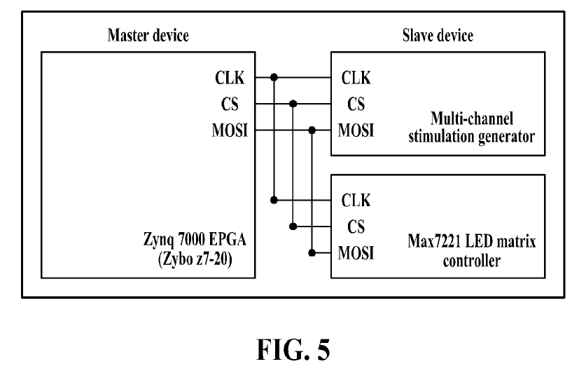

FIG. 5 is a block diagram illustrating a control system of a hybrid retinal stimulation device, according to an embodiment.

Referring to FIG. 5, a hybrid retinal stimulation device (e.g., the hybrid retinal stimulation device 100 of FIG. 1) may precisely control and synchronize, between electrical stimulation and optical stimulation, a start timing, duration time, intensity, and the like thereof by using an SPI communication structure based on a master-slave structure.

More specifically, the hybrid retinal stimulation device may include an FPGA-based master device and two slave devices (e.g., a multi-channel stimulation generator and an LED matrix controller) connected to the master device.

The master device may communicate with the slave devices via an SPI protocol and may control and synchronize, between the electrical stimulation and the optical stimulation, at least one parameter of the start timing, duration time, and intensity thereof. Here, the SPI protocol may be performed through a reference signal (clock; CLK) for communication synchronization, a selection signal (chip select; CS) for selecting each slave device, and a transmission signal (master out slave in; MOSI) for data transmission from the master device to the slave device.

Among the two slave devices, a first slave device may function as a multi-channel stimulation generator, which may drive a stimulation electrode of the hybrid retinal stimulation device. The first slave device may receive a transmission signal from the FPGA of the master device via the SPI protocol to set at least one of an intensity, a frequency, a pulse width, or a duration time of the electrical stimulation (e.g., voltage/current) and control the stimulation electrode to output electrical stimulation to the retinal cells.

A second slave device among the two slave devices may function as an LED matrix controller, which may drive a micro LED array of the hybrid retinal stimulation device. The second slave device may receive a transmission signal from the FPGA of the master device via an SPI protocol to set at least one of an intensity, a blinking cycle, or a duration time of the optical stimulation and may control the micro LED array to output the optical stimulation to the retinal cells.

The hybrid retinal stimulation device of the present disclosure may improve both neural stimulation efficiency and safety of electrical stimulation and optical stimulation by minimizing a stimulation timing error between the electrical stimulation and the optical stimulation using a single reference signal through the SPI communication structure based on a master-slave structure.

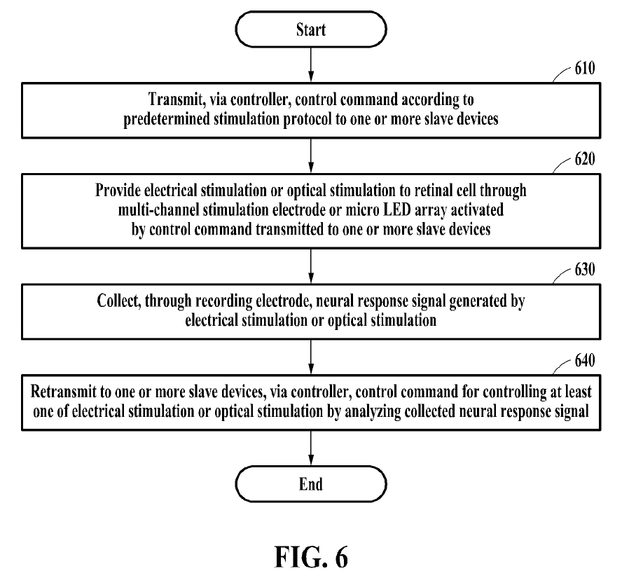

FIG. 6 is a flowchart illustrating a method of providing hybrid stimulation of a hybrid retinal stimulation device, according to an embodiment. In an example, at least one of the operations of FIG. 6 may be performed simultaneously or in parallel with another, and the order of the operations may be changed. In addition, at least one of the operations may be omitted, or another operation may be additionally performed. The operations illustrated in FIG. 6 may be performed by at least one component of the hybrid retinal stimulation device (e.g., the hybrid retinal stimulation device 100 of FIG. 1).

In operation 610, the hybrid retinal stimulation device may transmit a control command according to a predetermined stimulation protocol to one or more slave devices via a controller implemented based on an FPGA and configured to operate as a master device. Here, the control command may include information regarding a start timing and a pulse width of stimulation, an intensity, a frequency, and a duration time of electrical stimulation (e.g., voltage/current), an intensity, a blinking cycle, and a duration time of optical stimulation (e.g., brightness), a stimulation target channel, or the like.

In operation 620, the hybrid retinal stimulation device may provide electrical stimulation or optical stimulation to retinal cells through a multi-channel stimulation electrode or a micro LED array activated by the control command transmitted to the one or more slave devices. Electrical stimulation and optical stimulation may be provided simultaneously or with a time difference within one stimulation cycle, thereby realizing a neural stimulation effect of a hybrid stimulation method.

In operation 630, the hybrid retinal stimulation device may collect, through a recording electrode, a neural response signal (e.g., a spike signal, an action potential, etc.) generated by the provided electrical stimulation or the provided optical stimulation.

In operation 640, the hybrid retinal stimulation device may retransmit to the one or more slave devices, via the controller, a control command for controlling at least one of the electrical stimulation or the optical stimulation by analyzing the collected neural response signal.

According to an embodiment, the controller may generate a control signal for controlling at least one of the intensity, the frequency, a pulse width, or the duration time of the electrical stimulation by analyzing a neural response signal measured through the recording electrode. For example, when it is determined that, as a result of the analysis of the neural response signal, the recording electrode did not detect any neural response signal for particular stimulation, the controller may control an electrical pulse for electrical stimulation for a next cycle to have a wider pulse width or a stronger current value than before.

According to another embodiment, the controller may generate a control signal for controlling at least one of the intensity, the blinking cycle, or the duration time of the optical stimulation by analyzing the neural response signal measured through the recoding electrode. For example, when it is determined that, as a result of the analysis of the neural response signal, the recording electrode has detected an excessive neural signal for particular stimulation, the controller may control an optical pulse for optical stimulation for a next cycle to have a lower intensity or a shorter duration time than before.

The hybrid retinal stimulation device may improve stimulation efficiency and minimize tissue damage by controlling an output of the multi-channel stimulation electrode or the micro LED array, using the control signal generated by the controller.

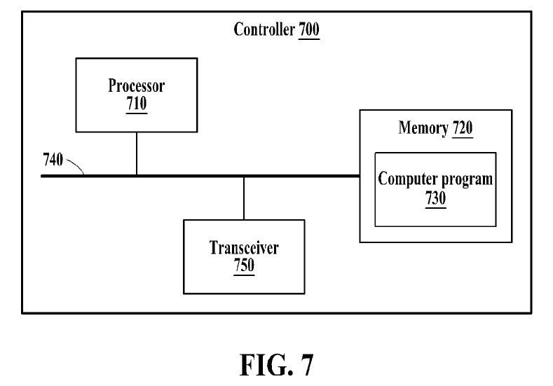

FIG. 7 is a diagram illustrating a controller included in a hybrid retinal stimulation device, according to an embodiment.

Referring to FIG. 7, a controller 700 may include one or more processors 710 and a memory 720 that may load or store a computer program 730 executed by the processor 710. The processor 710 and the memory 720 may be connected to each other via a communication link (e.g., a bus) 740. Optionally, the controller 700 may further include a transceiver 750, which may be used for data exchange, such as transmission and/or reception of data between the controller 700 and another electronic device (e.g., a slave device). The components included in the controller 700 of FIG. 7 are only examples, and one of ordinary skill in the art to which the present disclosure pertains may understand that other generally used components may further be included in addition to the components illustrated in FIG. 7.

The processor 710 may control the overall operation of each component of the controller 700. The processor 710 may be implemented as circuitry (e.g., processing circuitry) such as a system on chip (SoC) or an integrated circuit (IC). The processor 710 may include one or more processors. For example, the processor 710 may include a combination of one or more processors, such as a central processing unit (CPU), a microprocessor unit (MPU), a microcontroller unit (MCU), a graphics processing unit (GPU), a neural processing unit (NPU), a digital signal processor (DSP), an application processor (AP), a communication processor (CP), or any other processors well known in the technical field of the disclosure. In addition, the processor 710 may perform an operation on the computer program 730 or at least one application to execute methods and/or operations according to various examples of the present disclosure.

The memory 720 may store one or a combination of two or more of various pieces of data, instructions, and information used by a component (e.g., the processor 710) included in the controller 700. The memory 720 may include volatile memory and/or non-volatile memory.

The computer program 730 may include one or more actions through which the methods and/or operations described herein according to various embodiments are implemented and may be stored in the memory 720 as software. In this case, the action may correspond to an instruction that is implemented in the computer program 730. For example, the computer program 730 may include instructions for performing an operation of transmitting a control command according to a predetermined stimulation protocol to one or more slave devices and an operation of retransmitting to the one or more slave devices, a control command for controlling at least one of the electrical stimulation or the optical stimulation by analyzing a neural response signal collected through a recording device.

When the computer program 730 is loaded to the memory 720, the processor 710 may execute a plurality of operations to implement the computer program 730 and may thus perform the methods/operations according to various embodiments of the present disclosure.

The communication link 740 may include a path to transmit at least one of various pieces of data, instructions, and information among components included in the controller 700. The communication link 740 may be, for example, a peripheral component interconnect (PCI) bus or an extended industry standard architecture (EISA) bus. However, these types of buses are only an example, and embodiments are not limited thereto. For example, in FIG. 7, a bus is illustrated by a single line for ease of description, but a plurality of buses or various types of buses may be included.

The embodiments described herein may be implemented using a hardware component, a software component, and/or a combination thereof. For example, a processing device may be implemented using one or more general-purpose or special-purpose computers, such as, for example, a processor, a controller, an arithmetic logic unit (ALU), a DSP, a microcomputer, an FPGA, a programmable logic unit (PLU), a microprocessor, or any other device capable of responding to and executing instructions in a defined manner. The processing device may run an operating system (OS) and one or more software applications that run on the OS. The processing device may also access, store, manipulate, process, and create data in response to execution of the software. For purpose of simplicity, the processing device is described as singular. However, one of ordinary skill in the art will appreciate that a processing device may include multiple processing elements and/or multiple types of processing elements. For example, the processing device may include a plurality of processors, or a single processor and a single controller. In addition, a different processing configuration is possible, such as one including parallel processors.

The software may include a computer program, a piece of code, an instruction, or one or more combinations thereof, to independently or collectively instruct or configure the processing device to operate as desired. The software and/or data may be stored in any type of machine, component, physical or virtual equipment, or computer storage medium or device for the purpose of being interpreted by the processing device or providing instructions or data to the processing device. The software may also be distributed over network-coupled computer systems so that the software is stored and executed in a distributed fashion. The software and data may be stored in a non-transitory computer-readable recording medium.

The methods according to the embodiments may be recorded in non-transitory computer-readable media including program instructions to implement various operations of the embodiments. The media may also include the program instructions, data files, data structures, and the like alone or in combination. The program instructions recorded on the media may be those specially designed and constructed for the embodiments, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as floptical disks; and hardware devices that are specially configured to store and perform program instructions, such as read-only memory (ROM), random-access memory (RAM), flash memory, and the like. Examples of program instructions include both machine code, such as those produced by a compiler, and files containing high-level code that may be executed by the computer using an interpreter.

The above-described hardware devices may be configured to act as one or more software modules in order to perform the operations of the above-described embodiments, or vice versa.

Although the embodiments have been described with reference to the limited number of drawings, one of ordinary skill in the art may apply various technical modifications and variations based thereon. For example, suitable results may be achieved if the described techniques are performed in a different order and/or if components in a described system, architecture, device, or circuit are combined in a different manner and/or replaced or substituted by other components or their equivalents.

Therefore, other implementations, other embodiments, and equivalents to the claims are also within the scope of the following claims.

What is claimed is:

1. A hybrid retinal stimulation device, the device comprising:
a glass substrate in which a through hole for providing electrical stimulation and optical stimulation to retinal cells is formed;
a multi-channel stimulation electrode arranged in the through hole of the glass substrate and configured to provide the electrical stimulation to the retinal cells;
a multi-channel recording electrode arranged in the through hole of the glass substrate to measure a neural response signal generated from the retinal cells while arranged at a position spaced apart by a determined distance from the multi-channel stimulation electrode;
a micro light-emitting diode (LED) array arranged in a lower portion of the glass substrate and configured to provide the optical stimulation to the retinal cells from between the multi-channel stimulation electrode and the multi-channel recording electrode; and
a controller configured to control synchronization between the electrical stimulation and the optical stimulation by independently controlling an operation of the multi-channel stimulation electrode and the micro LED array.

2. The device of claim 1, wherein
the controller is implemented based on a field-programmable gate array (FPGA) and configured to operate as a master device.

3. The device of claim 2, wherein the controller is configured to:
communicate with one or more slave devices, using serial peripheral interface (SPI) communication, and independently control the multi-channel stimulation electrode providing the electrical stimulation to the retinal cells or the micro LED array providing the optical stimulation to the retinal cells.

4. The device of claim 1, wherein the controller is configured to:
control at least one of an intensity, a frequency, a pulse width, or a duration time of the electrical stimulation by analyzing a neural response signal measured through the multi-channel recording electrode.

5. The device of claim 1, wherein the controller is configured to:
control at least one of an intensity, a blinking cycle, or a duration time of the optical stimulation by analyzing a neural response signal measured through the multi-channel recording electrode.

6. The device of claim 1, wherein the multi-channel stimulation electrode and the multi-channel recording electrode are formed by:
forming a pillar-shaped vertical through structure on a silicon substrate and bonding a glass-based dielectric on the silicon substrate on which the vertical through structure is formed, through a deep silicon (Si) etching process and an anodic bonding process, respectively;
generating a glass substrate by filling an empty area of the silicon substrate with the glass-based dielectric after lowering viscosity of the dielectric by heating the dielectric, and removing the dielectric remaining after filling the empty area based on an upper surface of the silicon substrate, through a glass reflow process and a chemical mechanical polishing (CMP) process, respectively;
forming a micro-pillar structure by selectively etching a partial area of the silicon substrate through the deep silicon etching process while the silicon substrate is arranged in an inverted orientation;
forming a pointed electrode structure by gradually etching exposed silicon regions of the micro-pillar structure through a wet etching process, and forming a metal pattern on the pointed electrode structure through a metal patterning process; and
arranging electrodes on a lower surface of the glass substrate and performing dicing through an electrode arrangement process and a dicing process, respectively.

7. The device of claim 1, wherein the micro LED array is formed by:
patterning a metal layer on a substrate according to a predetermined electrode structure and subsequently adding an insulating layer between the patterned metal layer through a metal patterning process and an insulation process, respectively;
depositing a chromium (Cr) layer and a gold (Au) layer on top of the metal layer and the insulating layer through a sputtering process;
coating a photosensitive agent on top of the chromium layer and the gold layer and subsequently plating a copper (Cu) layer thereon through a copper electroplating process;
forming a solder bump on top of the copper layer through a solder bump electroplating process; and
placing an individual micro LED onto the solder bump through a pick & place process.

8. An operating method of a hybrid retinal stimulation device, the operating method comprising:
transmitting a control command according to a predetermined stimulation protocol to one or more slave devices via a controller implemented based on a field-programmable gate array (FPGA) and configured to operate as a master device;
providing electrical stimulation or optical stimulation to retinal cells through a multi-channel stimulation electrode or a micro light-emitting diode (LED) activated by the control command transmitted to the one or more slave devices;
collecting, through a recording electrode, a neural response signal generated by the provided electrical stimulation or the provided optical stimulation; and
retransmitting to the one or more slave devices, via the controller, a control command for controlling at least one of the electrical stimulation or the optical stimulation by analyzing the collected neural response signal.

9. The operating method of claim 8, wherein the retransmitting of the control command comprises:
controlling at least one of an intensity, a frequency, a pulse width, or a duration time of the electrical stimulation by analyzing a neural response signal measured through the recording electrode.

10. The operating method of claim 8, wherein the retransmitting of the control command comprises:
controlling at least one of an intensity, a blinking cycle, or a duration time of the optical stimulation by analyzing a neural response signal measured through the recording electrode.

11. A non-transitory computer-readable storage medium storing instructions that, when executed by a processor, cause the processor to perform the method of claim 8.
